(19) United States
(12) Patent Application Publication
(10) Pub. No.: US 2024/0189264 A1
(43) Pub. Date: Jun. 13, 2024

(54) Method for Assessing Atenolol with Precision

(71) Applicants: Firoz Anwar, Jeddah (SA); Moayad Mustafa Hejazi, Jeddah (SA); Mirza R. Baig, Dubai (AE); Fahad A. Al-Abbasi, Jeddah (SA); Ryan Adnan Sheikh, Jeddah (SA); Naif A. R. Almalki, Jeddah (SA); Vaseem Ahmad Ansari, Lucknow (IN); Azizah Salim Bawadood, Al-Kharj (SA)

(72) Inventors: Firoz Anwar, Jeddah (SA); Moayad Mustafa Hejazi, Jeddah (SA); Mirza R. Baig, Dubai (AE); Fahad A. Al-Abbasi, Jeddah (SA); Ryan Adnan Sheikh, Jeddah (SA); Naif A. R. Almalki, Jeddah (SA); Vaseem Ahmad Ansari, Lucknow (IN); Azizah Salim Bawadood, Al-Kharj (SA)

(21) Appl. No.: 18/416,116

(22) Filed: Jan. 18, 2024

(51) Int. Cl. A61K 31/165; A61K 47/20; A61K 47/26; A61K 47/38

(52) U.S. Cl. CPC A61K 31/165; A61K 47/20; A61K 47/26; A61K 47/38

(57) ABSTRACT

The present invention relates to a method for assessing Atenolol with precision. A pharmaceutical composition comprising: a predefined amount of Atenolol or pharmaceutically acceptable salts thereof; and a combination of pharmaceutical excipients suitable for oral administration, wherein the method for tailoring the treatment of hypertension comprises of determining the levels of C-reactive protein, blood pressure, blood urea, and HbA1c in hypertensive patients; stratifying patients into distinct subgroups based on the identified biomarker levels; and customizing antihypertensive treatment plans for each subgroup, wherein treatment may include, but is not limited to, beta blockers such as Atenolol.

### FIELD OF THE INVENTION

The present invention relates to the field of biomedical compositions and processes. In particular, the present invention relates to a method for assessing Atenolol with precision.

### BACKGROUND OF THE INVENTION

Among all causes of death and morbidly, hypertension is one of the major reasons across the globe. Hypertension accounts for major reason of mortality and morbidity globally—a reason that can be controlled for cardiovascular disease (CVD; comprising of chronic kidney disease, (CKD), heart failure, myocardial infarction, stroke, atrial fibrillation, peripheral artery disease, cognitive impairment and other coronary heart diseases. However, it is a leading consequence for cardiovascular disease and death, which is due to smoking, obesity, and diabetes mellitus. The most significant predictor of early mortality and cardiovascular impairment is hypertension, causing mark or significant economic burden both on human capital and health care expenses.

A 13.5 percent of all early mortality (7.6 million), 47 percent of ischemic heart disease, and 54 percent of strokes solely attribute to hypertension, globally. Medical practitioners must be aware of the potential cause for high blood pressure in this population given the level of disease burden present. This pattern of rising hypertension-related fatalities is expected to continue unless initiatives are implemented to spread awareness amongst Saudi population. The concept of pre-hypertension is characterized by 120 to 139 mm of Hg as systolic blood pressure and/or 80 to 89 mm of Hg as diastolic blood pressure is introduced in 7th report of National committee on prevention, detection, evaluation and treatment of high blood pressure.

Beta adrenergic blockers such as propranolol, metoprolol or atenolol are typically the first line of drugs in treatment of hypertension. They have negative chronotropic and negative inotropic effects. Moreover, atenolol is one of the most widely used second generation cardio selective β1-blockers used as reference drug in randomized controlled studies on hypertension Atenolol selectively act on β1 adrenergic receptors rich on vascular smooth muscles and heart, interfering with positive ionotropic and chronotropic actions of neurotransmitters specially the nor adrenaline, and other catecholamines including isoproterenol and adrenaline thereby blocking the sympathetic action on these receptors. β1 blocking activity retards heart rate, drops blood pressure and reduces myocardial contractility. However, contrary to positive use atenolol can increase the end diastolic pressure and left ventricular fiber length in cardiac failure patients that results in increased oxygen demand by cardiac tissues.

It is well established at higher doses atenolol competitively block β2 adrenergic receptors located in bronchial and vascular smooth muscle, with no intrinsic sympathomimetic activity or membrane stabilizing effects. Atenolol, does not interfere with CNS activity due to low lipid solubility, resulting reduced brain penetration and few adverse neurological effects. Since atenolol possesses β adrenergic antagonist potential, hence it is known to increase the AV node's refractory period. Action on AV node has led this drug to treat the supraventricular tachycardia and prevents the paroxysmal attacks of atrial fibrillation.

However, the correct dosage of atenolol has not been identified in the literature that is effective enough for all age group to treat hypertension.

Therefore, there exists a need to develop a composition and a method for developing and evaluating Atenolol dosage and identify a best fixed dose of atenolol that is effective in all age group and all weight patients irrespective of the nature of hypertension with other associated parameters.

The technical advancements disclosed by the present invention overcome the limitations and disadvantages of existing and conventional systems and methods.

### SUMMARY OF THE INVENTION

The present invention generally relates to a composition and a process for evaluating Atenolol.

Another object of the present invention is to identify a fixed dose for Atenolol; and

Yet another object of the present invention is to identify a dosage effective for all age group and all weight patients irrespective of the nature of hypertension with other associated parameters.

In an embodiment, a pharmaceutical composition comprises of a predefined amount of Atenolol or pharmaceutically acceptable salts thereof; and a combination of pharmaceutical excipients suitable for oral administration.

In an embodiment, a method for tailoring the treatment of hypertension, comprising the steps of:
a first step of determining the levels of C-reactive protein, blood pressure, blood urea, and HbA1c in hypertensive patients;
a second step of stratifying patients into distinct subgroups based on the identified biomarker levels; and
a third step of customizing antihypertensive treatment plans for each subgroup, wherein treatment may include, but is not limited to, beta blockers such as Atenolol.

To further clarify the advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present invention will become better understood when the following detailed description is read with reference to the accompanying drawings in which like characters represent like parts throughout the drawings, wherein:

FIG. 1 illustrates a flow diagram of a method for tailoring the treatment of hypertension using a pharmaceutical composition comprising,

FIG. 2 illustrates a tabular representation of demographic Data of the participant with Atenolol,

FIG. 3 illustrates a tabular representation of Vital Signs & Laboratory Data of the participant with Atenolol,

FIGS. 4a and 4b illustrates a graphical representation of a) Blood Pressure status in Age groups, high BP is observed in patient above the age of 50 years and b) Blood Pressure status in male female genders, irrespective of gender difference, both the groups having high BP,

FIG. 5 illustrates a graphical representation of HbA1c Variations in Gender, Males are dominant of high HBA1c, and

FIG. 6 illustrates a tabular representation of comparison of Lab data & demographics: ANOVA test P− value <0.05: Males tends to have upper level of CRP, Creatinine and BUN. Participants with more than 60 years have upper level of CRP, Creatinine and BUN.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present invention. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present invention will be described below in detail with reference to the accompanying drawings.

A pharmaceutical composition comprising of a predefined amount of Atenolol or pharmaceutically acceptable salts thereof; and a combination of pharmaceutical excipients suitable for oral administration.

In an embodiment, the predefined amount of Atenolol is adjusted based on biomarker profiling, wherein the biomarkers include C-reactive protein, blood pressure, blood urea, and HbA1c levels.

In an embodiment, the predefined amount of Atenolol for hypertensive patients classified as obese or overweight based on their Body Mass Index (BMI) is 50 mg.

In an embodiment, the predefined amount of Atenolol for hypertensive patients aged 60 years and above with significantly elevated C-reactive protein levels is 75 mg.

In an embodiment, the predefined amount of Atenolol for hypertensive patients with elevated systolic blood pressure and high HbA1c levels is 150 mg.

In an embodiment, the predefined amount of Atenolol for hypertensive patients aged 60 years and above with significantly elevated creatinine and BUN levels is 125 mg.

In an embodiment, the pharmaceutically acceptable salts of Atenolol include Atenolol hydrochloride, Atenolol sulfate, Atenolol acetate, or Atenolol citrate.

In an embodiment, the pharmaceutically acceptable salts of Atenolol include Atenolol hydrochloride, Atenolol sulfate, Atenolol acetate, or Atenolol citrate.

In an embodiment, the pharmaceutical excipients are selected from a group comprising microcrystalline cellulose, lactose monohydrate, magnesium stearate, sodium lauryl sulfate.

FIG. 1 illustrates a flow diagram of a method (100) for tailoring the treatment of hypertension using a pharmaceutical composition comprising the steps of:
Step (102) discloses about determining the levels of C-reactive protein, blood pressure, blood urea, and HbA1c in hypertensive patients;
Step (104) discloses about stratifying patients into distinct subgroups based on the identified biomarker levels; and
Step (106) discloses about customizing antihypertensive treatment plans for each subgroup, wherein treatment may include, but is not limited to, beta blockers such as Atenolol.

In an embodiment, the method further comprising of identifying hypertensive patients classified as obese or overweight; and administering 100 mg Atenolol cautiously to the identified obese or overweight patients.

In an embodiment, the method further comprising: assessing systolic blood pressure and HbA1c levels in hypertensive patients; and adjusting the dosage of Atenolol, specifically 100 mg, based on the identified elevated systolic blood pressure and high HbA1c levels.

In an embodiment, the method further comprising: analysing C-reactive protein levels in hypertensive patients aged 60 years and above; and revalidating the use of 100 mg Atenolol in hypertensive patients with significantly elevated C-reactive protein levels, particularly in the elderly.

In an embodiment, the method further comprising: evaluating creatinine and BUN levels in hypertensive patients aged 60 years and above; and re-evaluating the use of 100 mg Atenolol in hypertensive patients with significantly elevated creatinine and BUN levels, especially in the elderly.

In an exemplary embodiment, the data is retrieved from medical information database of patients registered as an out/indoor patients at cardiology department. The inclusion criteria for enrollment involves all patients who are clinically diagnosed through cardiological assessment including echo and biochemical laboratory analysis with hypertrophic cardiomyopathy. Patients having only severe hypertension, terminal illness are excluded from the study. This sample size of 131 is calculated by keeping the prevalence of HCM as 0.03-0.2%, 95% confidence interval and 5% margin of error. Information regarding Clinical history, age, gender, comorbidity, symptoms, clinical assessment of results along with ECG and associated factors are well documented in a form of questionnaire sheets. Furthermore, information on weight, blood group, BP, pulse and hemoglobulin (Hb) was also recorded from the health registry with no extra contact with patients for blood sample collection.

The assumed odd ratio is considered as 5 and expected proportion exposed in control groups is 20%. The intention is to have higher sample size but the patients are not only for hypertension treatment, most of them are having associated diseases including diabetes and hyperthyroidism, making the restriction of sample size to 131 only. After obtaining due clearance patient record and information is extracted from health facility registry both from electronic and paper files with all relevant information.

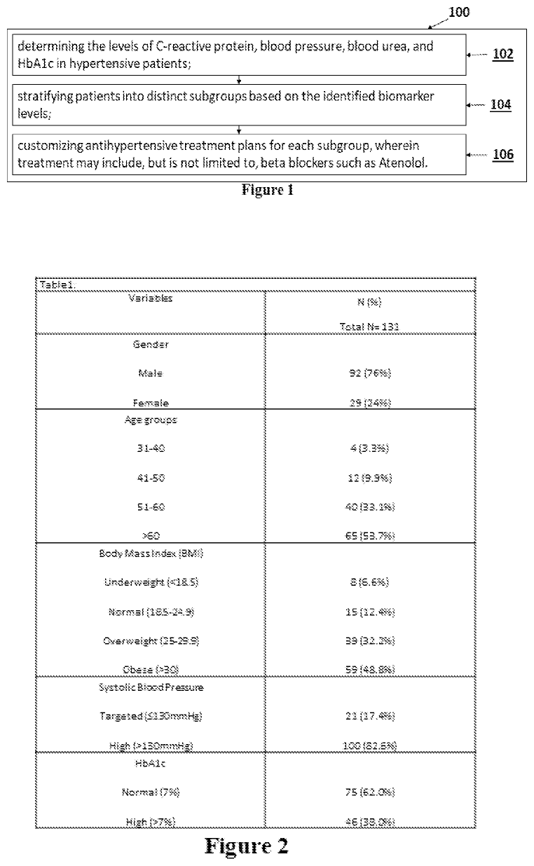

FIG. 2 illustrates a tabular representation of demographic Data of the participant with Atenolol. 131 patients are enrolled respectively, 29 women (24%) and 92 men (76%). The age variation of patients ranged between 31 to 65 years. The remaining parameters assessed before the start of the study is recorded.

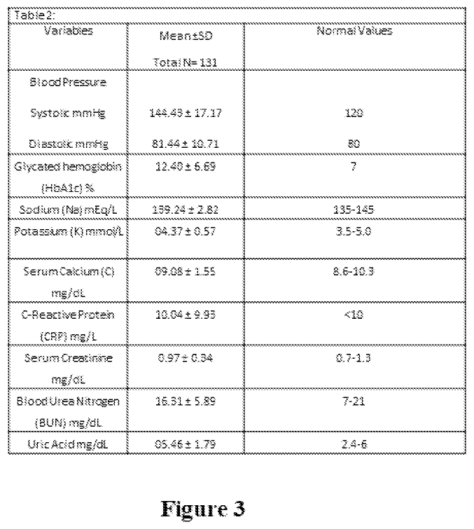

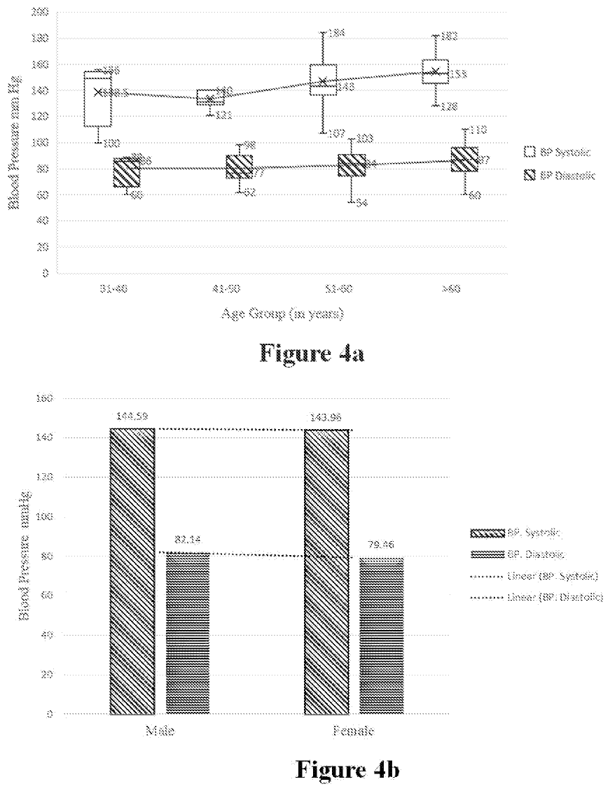

FIG. 3 illustrates a tabular representation of Vital Signs & Laboratory Data of the participant with Atenolol. The vital parameters of all participants consuming single dose of atenolol and considered in the present invention is recorded from the collected data it is noted that Glycated hemoglobin (HbA1c) and C-Reactive Protein (CRP) were found beyond the normal range.

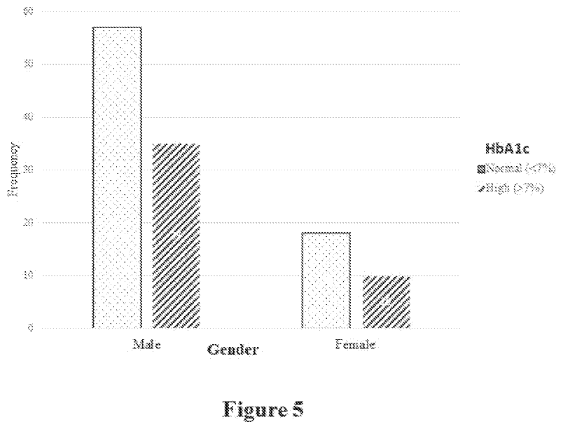

FIGS. 4a and 4b illustrates a graphical representation of a) Blood Pressure status in Age groups, high BP is observed in patient above the age of 50 years and b) Blood Pressure status in male female genders, irrespective of gender difference, both the groups having high BP.

The systolic blood pressure of patients under observation ranged from 100 to 182 mm of Hg (average or mean 144.43 f 17.17), whereas the diastolic blood pressure is fluctuating from 54 to 110 mm of Hg (average or mean 81.44 f 10.77). It is observed that blood pressure varied significantly among different age group. Patients above the age of 50 years have marked alteration both in systolic and diastolic blood pressure, both systolic and diastolic blood pressure are higher in the age groups above 50 years of age, irrespective of gender difference. Both male and female had high BP. No statistical difference is noted in blood pressure of male and female population.

FIG. 5 illustrates a graphical representation of HbA1c Variations in Gender, Males are dominant of high HBA1c. Galycated hemoglobin a marker for uncontrolled blood sugar is higher in the patients than the normal value. (Mean 12.40±6.69). The changes in HbA1c is analyzed with respect to age, gender, and Body Mass Index. It is observed that HbA1c level is higher in male compared with female, but also glycated hemoglobin was higher in the study subjects above age 60 years. Comparing the level of HbA1c with respect to BMI, obese populations have much higher level of glycated hemoglobin than underweight and normal BMI populations.

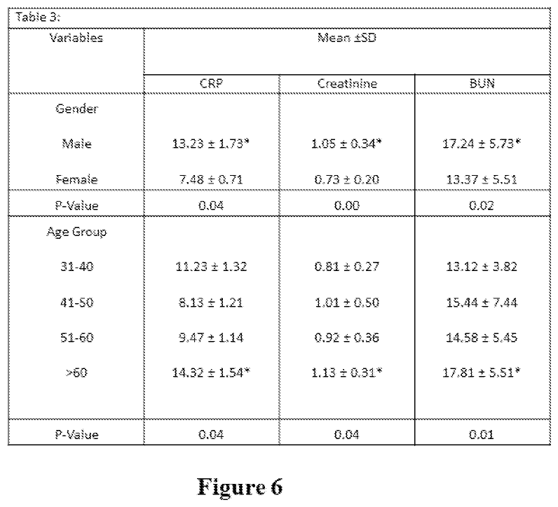

FIG. 6 illustrates a tabular representation of comparison of Lab data & demographics: ANOVA test P− value <0.05: Males tends to have upper level of CRP, Creatinine and BUN. Participants with more than 60 years have upper level of CRP, Creatinine and BUN.

The lab data and demographics are compared for various biochemical parameters, and it reeved that males have higher levels of C reactive protein (CRP), creatinine, and blood urea nitrogen (BUN) compared to females. Moreover, participants who have age above 60 years irrespective of gender show CRP level were 14.32±1.54*, Creatinine is 1.13±0.31* and blood urea are 17.81±5.51* that means CRP, Creatinine and BUN are higher.

Hypertension is a disorder that indicates serious attention, since it contributes to the development of coronary heart disease, stroke and vascular problems. The global incidence of diseases associated with hypertension is rising, possibly as a result of aging of the population, an epidemiological shift, industrialization, and growing age-specific rates of several chronic disorders. The Present invention clearly indicates, the blood pressure of the males is higher. Nevertheless, there is no noticeable gender variation in the overall occurrence of hypertension across the globe. The gender variations could be brought on by biological variations, but they could also be a result of other influencing variables such as sociodemographic, comorbidities, and obesity. BMI plays a vital role in maintaining the blood pressure, low BMI patients (18.5-22.9 kg/m²) had increased rates of hypertension as well; nonetheless, most earlier investigations had found that hypertension is more prevalent in the obese populations.

Further, it is well observed that hypertensive patients mostly respond to single dose of 100 mg atenolol on oral administration. C-reactive protein (CRP) level was significantly high in-patient taking atenolol single dose which leads to further health complications. CRP a biomarker for any inflammation or infection in human body, is now established and well documented that its level remains high in hypertensive patients. The increased CRP reflects the constant rise in acute phase in response of non-specific injury to systemic circulation parameters including, vascular injury, atherosclerosis, ischemic complications, angina and necrosis. Male subjects are more irresponsive toward atenolol treatment compared to women subjects as CRP, creatinine and BUN level is significantly high, and also subjects above age 60 years also have high levels of CRP, creatinine and BUN, considering the data summary, for such population alternative drugs like labetalol, combination therapy or another antihypertensive drug should take into consideration.

The present results are based on meta-analyses and other its role on other cardiovascular, alignments make it essential to revalidate the suitability of atenolol in a 100 mg fixed dose, as first line of therapeutic drug in treatment of hypertension for obese patients and potential reference drug in clinical trial output in large population study.

Beta blockers presented potential effectiveness against many cardiomyopathies including left ventricular hypertrophy, that depends upon the outflow tract of left ventricle. The results showed that C-reactive protein and HbA1c are higher in males in comparison to females. On the other hand, the findings also reported that C-reactive protein, blood pressure, blood urea level, and HbA1c are significantly high above the age of 60 years irrespective of gender.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that 5 may cause any benefit, advantage, or solution to occur or become more pronounced are not to be construed as a critical, required, or essential feature or component of any or all the claims.

We claim:

1. A pharmaceutical composition comprising:
a predefined amount of Atenolol or pharmaceutically acceptable salts thereof; and
a combination of pharmaceutical excipients suitable for oral administration.

2. The composition as claimed in claim 1, wherein the predefined amount of Atenolol is adjusted based on biomarker profiling, wherein the biomarkers include C-reactive protein, blood pressure, blood urea, and HbA1c levels.

3. The composition as claimed in claim 1, wherein the predefined amount of Atenolol for hypertensive patients classified as obese or overweight based on their Body Mass Index (BMI) is 50 mg.

4. The composition as claimed in claim 1, wherein the predefined amount of Atenolol for hypertensive patients aged 60 years and above with significantly elevated C-reactive protein levels is 75 mg.

5. The composition as claimed in claim 1, wherein the predefined amount of Atenolol for hypertensive patients with elevated systolic blood pressure and high HbA1c levels is 150 mg.

6. The composition as claimed in claim 1, wherein the predefined amount of Atenolol for hypertensive patients aged 60 years and above with significantly elevated creatinine and BUN levels is 125 mg.

7. The composition as claimed in claim 1, wherein the pharmaceutically acceptable salts of Atenolol include Atenolol hydrochloride, Atenolol sulfate, Atenolol acetate, or Atenolol citrate.

8. The composition as claimed in claim 1, wherein the pharmaceutically acceptable salts of Atenolol include Atenolol hydrochloride, Atenolol sulfate, Atenolol acetate, or Atenolol citrate.

9. The composition as claimed in claim 1, wherein the pharmaceutical excipients are selected from a group comprising microcrystalline cellulose, lactose monohydrate, magnesium stearate, sodium lauryl sulphate.

10. A method for tailoring the treatment of hypertension using compositing as claimed in claim 1, comprising the steps of:
determining the levels of C-reactive protein, blood pressure, blood urea, and HbA1c in hypertensive patients;
stratifying patients into distinct subgroups based on the identified biomarker levels; and
customizing antihypertensive treatment plans for each subgroup, wherein treatment may include, but is not limited to, beta blockers such as Atenolol.

11. The method as claimed in claim 10, further comprising:
identifying hypertensive patients classified as obese or overweight; and
administering 100 mg Atenolol cautiously to the identified obese or overweight patients.

12. The method as claimed in claim 10, further comprising:
assessing systolic blood pressure and HbA1c levels in hypertensive patients; and
adjusting the dosage of Atenolol, specifically 100 mg, based on the identified elevated systolic blood pressure and high HbA1c levels.

13. The method as claimed in claim 10, further comprising:
analyzing C-reactive protein levels in hypertensive patients aged 60 years and above; and
revalidating the use of 100 mg Atenolol in hypertensive patients with significantly elevated C-reactive protein levels, particularly in the elderly.

14. The method as claimed in claim 10, further comprising:
evaluating creatinine and BUN levels in hypertensive patients aged 60 years and above; and
re-evaluating the use of 100 mg Atenolol in hypertensive patients with significantly elevated creatinine and BUN levels, especially in the elderly.
